(19) United States
(12) Patent Application Publication
(10) Pub. No.: US 2026/0232557 A1
(43) Pub. Date: Aug. 13, 2026

(54) COLLABORATIVE CARE METHOD FOR SCALP HAIR FOLLICLES AND STRANDS

(71) Applicant: BIOMEDICAL RESEARCH ADVISORS LLC, HALLANDALE BEACH, FL (US)

(72) Inventor: SHIWEI HAN, Hollywood, FL (US)

(21) Appl. No.: 19/637,993

(22) Filed: Apr. 2, 2026

(51) Int. Cl. A61K 8/49; A61K 8/20; A61K 8/34; A61K 8/41; A61K 8/63; A61K 8/64; A61K 8/86; A61K 8/891; A61Q 5/02; A61Q 5/12; A61Q 19/00

(52) U.S. Cl. CPC A61K 8/4946; A61K 8/20; A61K 8/342; A61K 8/345; A61K 8/416; A61K 8/494; A61K 8/4953; A61K 8/498; A61K 8/63; A61K 8/64; A61K 8/86; A61K 8/891; A61Q 5/02; A61Q 5/12; A61Q 19/005; A61K 2800/48; A61K 2800/524; A61K 2800/884

(57) ABSTRACT

Provided is a collaborative care method for scalp hair follicles and strands, which includes using collaboratively rinse shampoo and conditioner. The shampoo includes a mild surfactant, a hair follicle support complex, a cationic conditioning polymer, and a microbiome regulator. The conditioner includes amino functional silicone oil, a fatty alcohol system, a cationic conditioning system, and a hair follicle support enhancing ingredients. The method utilizes a synergistic mechanism of cation deposition and carrier, lipid surfactant composite deposition, and geometric retention at hair follicle openings. Shampoo and conditioner form a synergistic method of continuous use. Shampoo is responsible for cleaning scalp hair follicles, improving hair follicle permeability, and achieving initial deposition of effective ingredients; as a deposition amplifier and stabilizer, hair conditioner fixes and co deposits effective ingredients through film-forming structure, reducing loss rate, stabilizing scalp microenvironment, and extending functional support time window.

### TECHNICAL FIELD

The present disclosure relates to the technical field of caring products and caring product compositions, and particularly relates to a low irritation, washable collaborative care method for scalp hair follicles and strands, suitable for daily cleaning, hair follicle support, scalp condition improvement, especially suitable for sensitive scalp, long hair curly hair care, and post-transplant repair scenarios, which can be used repeatedly for a long time; specifically, it is a collaborative care method for scalp hair follicles and strands.

### BACKGROUND

Hair loss, thinning hair, and suboptimal scalp health problems plague a wide range of people, and their causes involve multiple factors such as genetics, hormones, inflammation, mechanical stimulation, and environmental influences. The existing caring products have the following defects.

Conventional shampoo products have a short contact time with the scalp, usually lasting 1-2 minutes. The active ingredients lack a targeted deposition mechanism, and are almost completely lost after rinsing, rendering it difficult to form sustained functional support for hair follicles. They can only achieve basic cleaning or short-term smooth effects.

Products with hair follicle support function often rely on indwelling preparations or drug ingredients, such as minoxidil and oral anti androgen drugs, which have problems such as strong irritation, poor compliance, and contraindications, and are not suitable for long-term daily use.

Hair conditioner has a single function, focusing only on smooth hair tails and not participating in scalp and follicle care. It is functionally disrelated from shampoo and cannot form a synergistic care effect.

Conventional products have a contradiction between mildness and effectiveness, often sacrificing the concentration of active ingredients to reduce irritation, resulting in mildness but ineffectiveness, while high concentration products are prone to damaging the scalp barrier.

The screening criteria for raw material safety are low, and the dosage of preservatives is high. Long term use may cause cumulative risks such as scalp sensitization and barrier damage, rendering it difficult to adapt to sensitive scalp or postoperative care needs.

Based on the above reasons, a collaborative care method for scalp hair follicles and strands is provided.

The purpose of the present disclosure is to provide a collaborative care method for scalp hair follicles and strands to solve the problems raised in the background technology mentioned above.

To achieve the above objectives, the present disclosure provides the following technical solution: a collaborative care method for scalp hair follicles and strands, includes: using collaboratively rinse shampoo and conditioner; the shampoo includes a mild surfactant, a hair follicle support complex, a cationic conditioning polymer, and a microbiome regulator; the conditioner includes amino functional silicone oil, a fatty alcohol system, a cationic conditioning system, and a hair follicle support enhancing ingredient; and the method achieves a retention of effective ingredients in hair follicle units and adjacent areas after washing through a synergistic mechanism of cation deposition and carrier, lipid and surfactant composite deposition, and geometric retention at hair follicle.

In some embodiments of the present disclosure, a total amount of the mild surfactant in the shampoo is 10-35wt%, and the mild surfactant includes 3-25wt% of anionic surfactant and 1-20wt% of nonionic surfactant; a total amount of the hair follicle support complex is 0.5-10wt%, and the hair follicle support complex includes 0.01-2wt% of olfactory receptor agonist, a peptide component, a plant extract, and vitamin derivative.

In some embodiments of the present disclosure, the amino functional silicone oil has a content of 0.5-20wt% of the hair conditioner, a total amount of the fatty alcohol system is 2-20wt%, a total amount of the cationic conditioning system is 0.05-7wt%, and a pH is 4.0-6.0.

In some embodiments of the present disclosure, raw materials of the shampoo and conditioner have been screened with an EWG score of 0-2 and basically do not contain high-risk or endocrine disrupting ingredients.

In some embodiments of the present disclosure, the shampoo is applied to the scalp and massaged for 3-5 minutes before rinsing, the hair conditioner is applied and massaged for about 10 minutes before gently rinsing, and the hair root is smeared with scalp essence and absorbed.

In some embodiments of the present disclosure, the method improves appearance of the scalp, reduces hair shedding, and enhances hair density through a synergistic effect of cleaning, sedimentation, stabilization, and enhancement.

In some embodiments of the present disclosure, the essence liquid includes water, glycerin, butanediol, Procapil, Sandalore, caffeine, biotin, sodium hyaluronate, panthenol, phenoxyethanol, ethyl hexyl glycerol, and rosemary essential oil.

In some embodiments of the present disclosure, further include a freeze-dried pretreatment solution that is used synergy; the pretreatment solution is a freeze-dried powder containing low concentration salicylic acid 0.5wt%, disodium EDTA 0.1wt%, and freeze-drying protectant; the pretreatment solution is dissolved in 38 ℃ warm water before use, applied to the scalp and massaged for 1 minute before shampooing; the pretreatment solution is configured to gently dissolve hair follicle orifice plugs, open hair follicle channels, and greatly improve the absorption rate of effective ingredients in a subsequent shampoo and it is suitable for people with hair follicle blockage type hair shedding.

Compared with the existing technology, the beneficial effects of the present disclosure are:
1. quickly improve hair shedding phenomenon, users have significant improvement, significantly optimize scalp condition; the appearance of scalp erythema is reduced, the clarity of hair follicle opening is significantly improved, the visible amount of oil and porphyrin is reduced, and the scalp hydration and comfort are correspondingly improved;
2. enhancing hair quality: after long-term use, the part line width is reduced from 2.5-4.5mm to 1.2-2.5mm, and the hair density and appearance are significantly improved; length of new hair growth reaches 2.0-4.5cm, and the new hair is thicker and more evenly colored;
3. low risk adaptation for long-term use: the raw materials have been screened with an EWG score of 0-2, and the total anti-corrosion dose is only 1/10 of conventional products; there are no high-risk or endocrine disrupting ingredients, rendering it suitable for sensitive scalp, scalp inflammation, and postoperative population having been hair transplanted;
4. synergistic efficiency breakthrough: shampoo and conditioner form a closed loop, and conditioner prolongs the retention time of active ingredients by 2-3 times, reduces mechanical friction, and significantly improves the continuity of the care;
5. solving the problem of hair follicle blockage, targeting the issue of orifice plugs blocking the hair follicle caused by seborrheic hair shedding, a mild combination of salicylic acid and disodium EDTA is used to dissolve old waste plug and calcium magnesium ion deposits, improve the smoothness of the hair follicle opening, and improve the absorption barrier of effective ingredients from the source; which increases the deposition of effective ingredients in the hair follicles of subsequent shampoos and shortens the onset time.

### BRIEF DESCRIPTION OF DRAWINGS

FIG. 1 is a bar chart of a distribution of testers by age range according to the present disclosure.

FIG. 2 is a bar chart of baseline hair shedding level among testers of the present disclosure.

FIG. 3 is a bar chart of baseline part line visibility scores in the present disclosure.

FIG. 4 is a bar chart of average hair shedding count over time according to the present disclosure.

FIG. 5 is a bar chart of average hair shedding reduction in the present disclosure.

FIG. 6 is a bar chart of a distribution of shedding reduction at 2 weeks of the present disclosure.

FIG. 7 is a bar chart of reduction in part line width after 12 weeks of the present disclosure.

FIG. 8 is a bar chart of hair density improvement after 12 weeks of the present disclosure.

FIG. 9 is a bar chart of distributions of new hair growth length in the 12th week of the present disclosure.

FIG. 10 shows a bar chart of scalp hydration improvement after 4 weeks according to the present disclosure.

FIG. 11 is a bar chart of scalp comfort improvement after 4 weeks according to the present disclosure.

FIG. 12 is a bar chart showing a change in scalp itchiness after 4 weeks of the present disclosure.

FIG. 13 is a first comparison diagram of scalp and hair roots under a trichoscopy analysis before and after use of the present disclosure.

FIG. 14 is a second comparison diagram of scalp and root conditions under a trichoscopy analysis before and after use of the present disclosure.

FIG. 15 is a third comparison diagram of scalp and root conditions under a trichoscopy analysis before and after use of the present disclosure.

### DESCRIPTION OF EMBODIMENTS

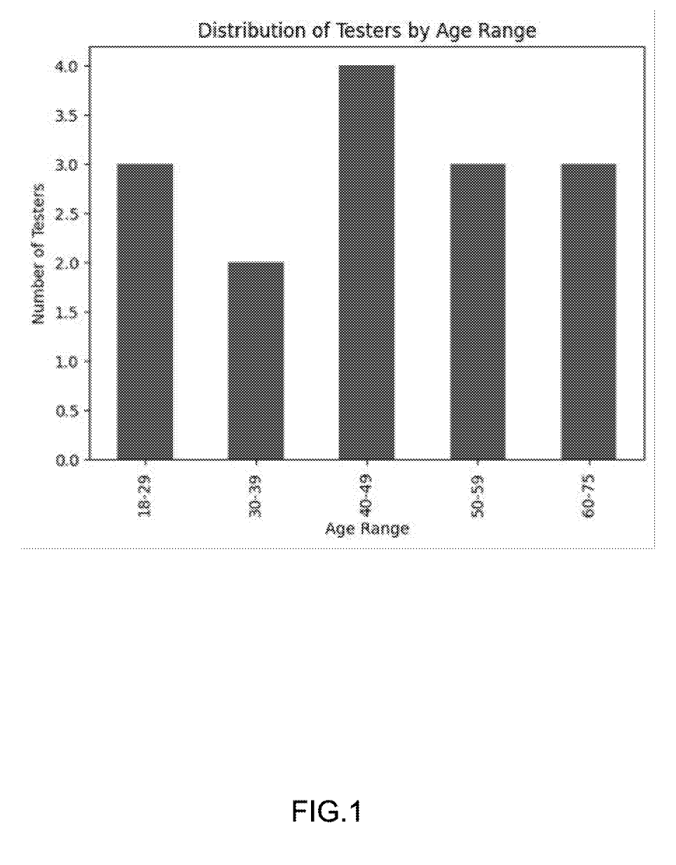

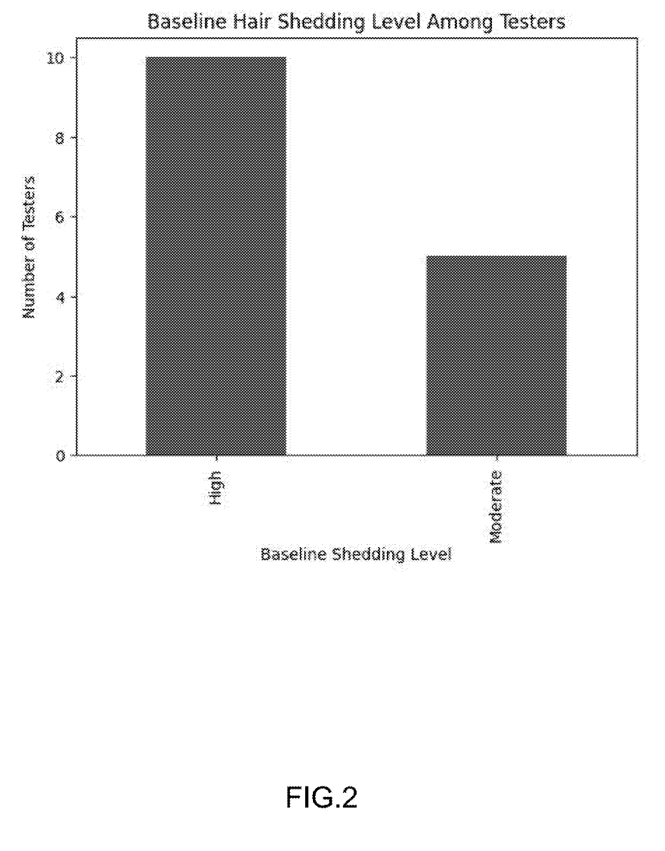

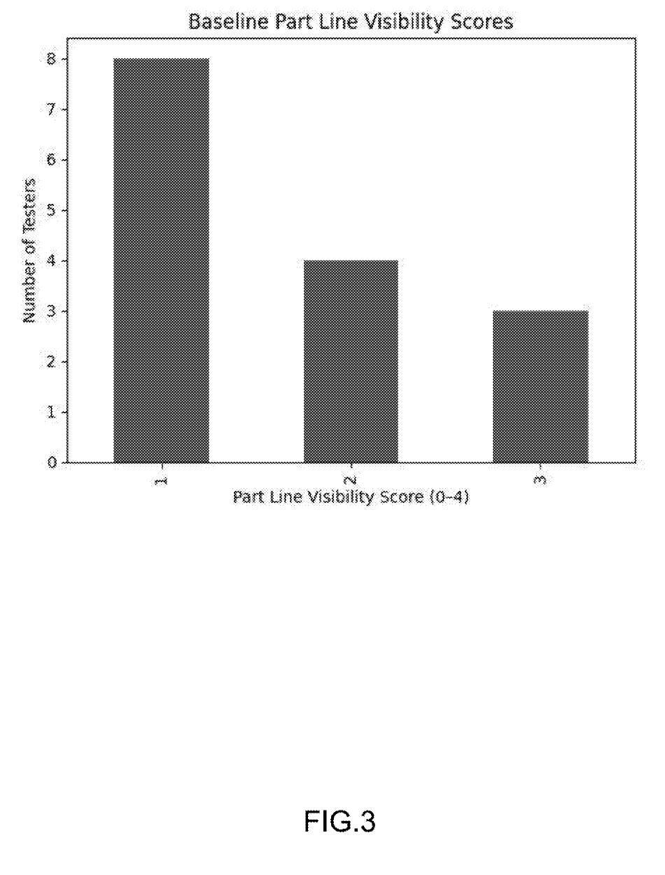

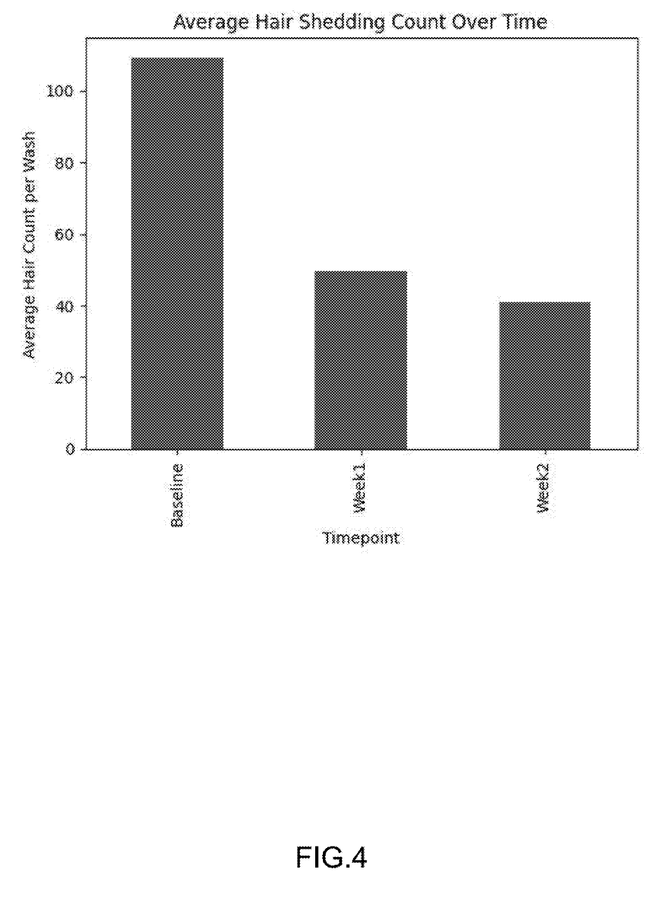

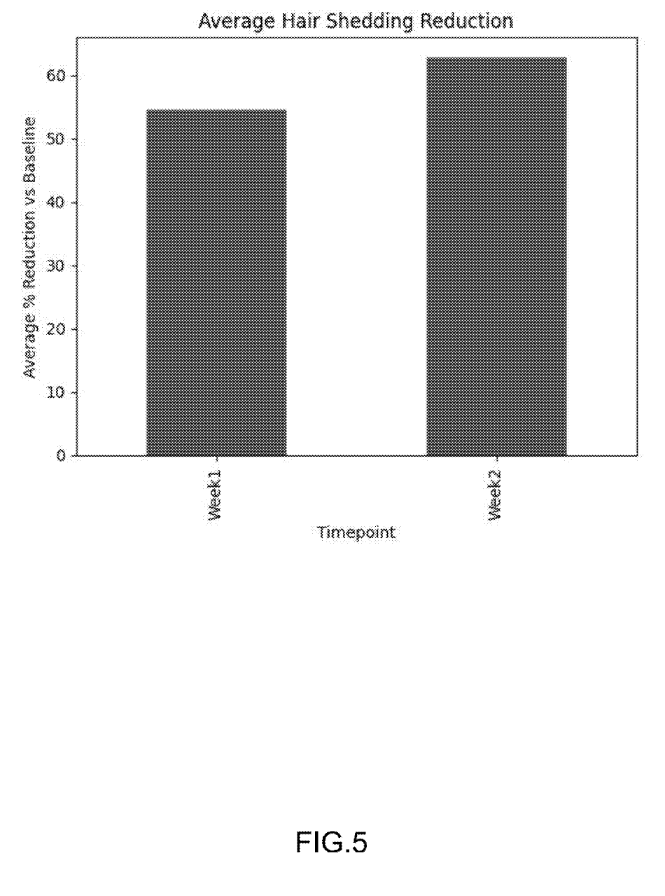

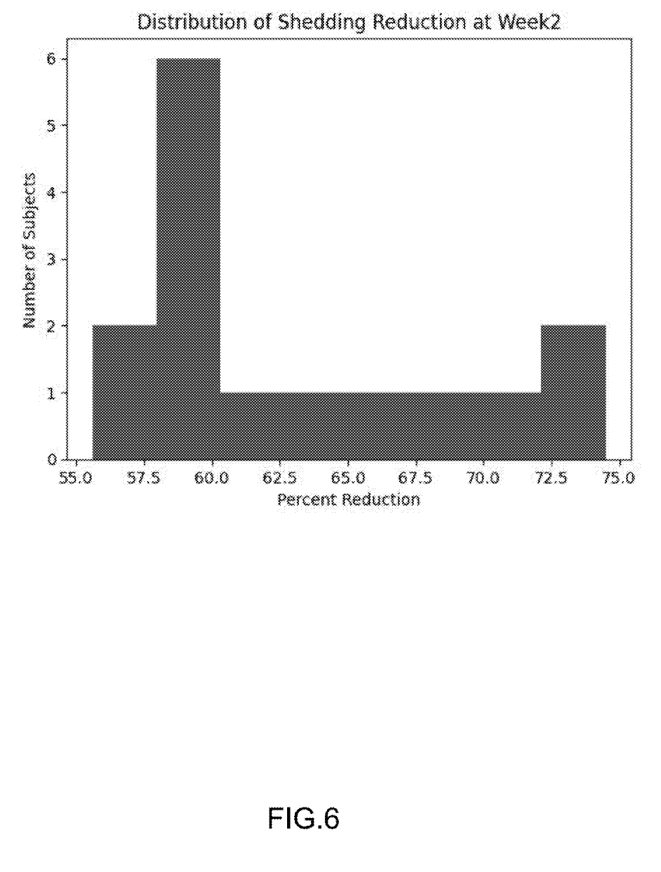

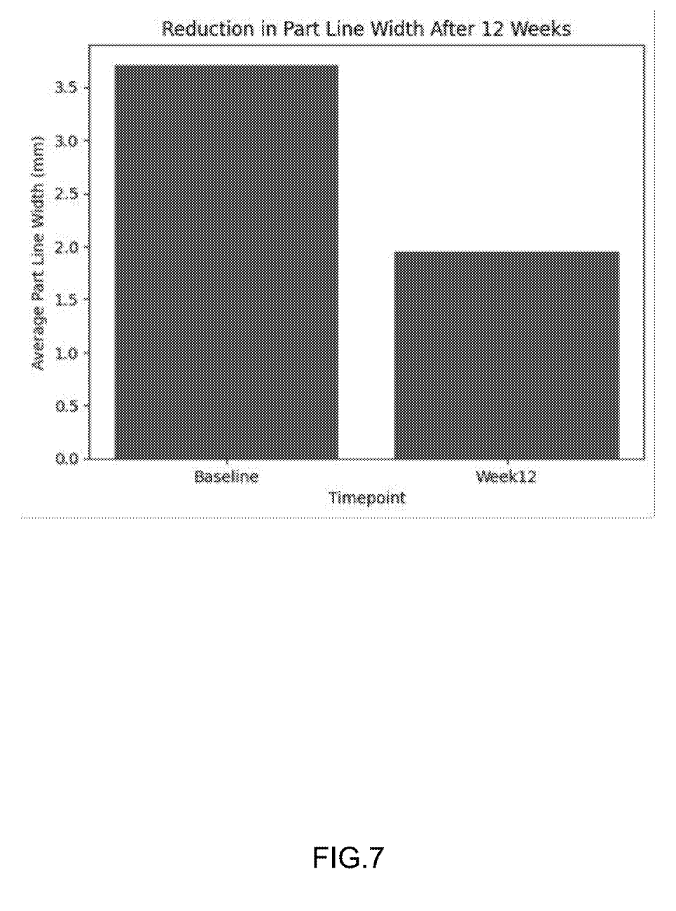

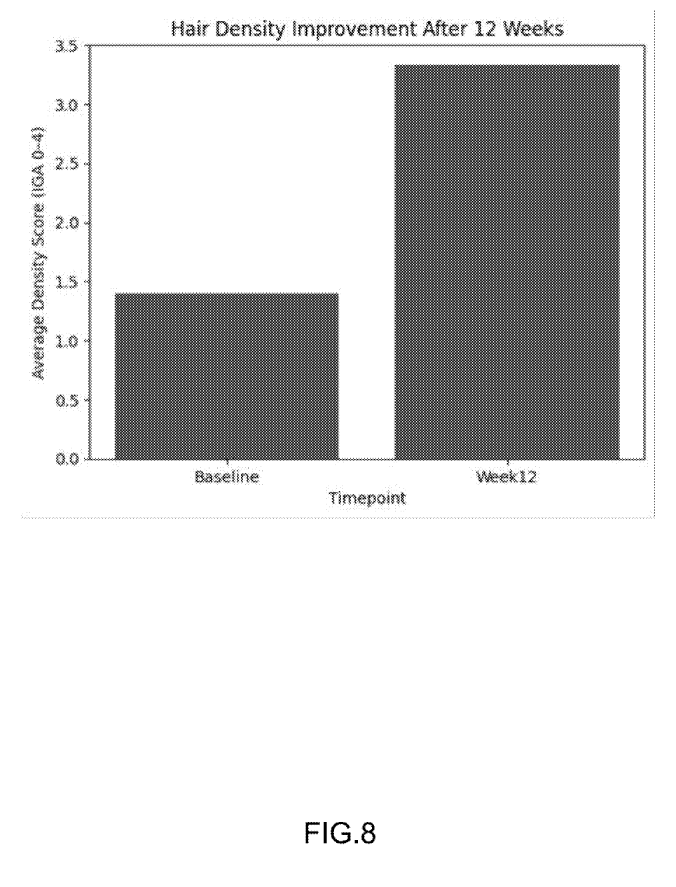

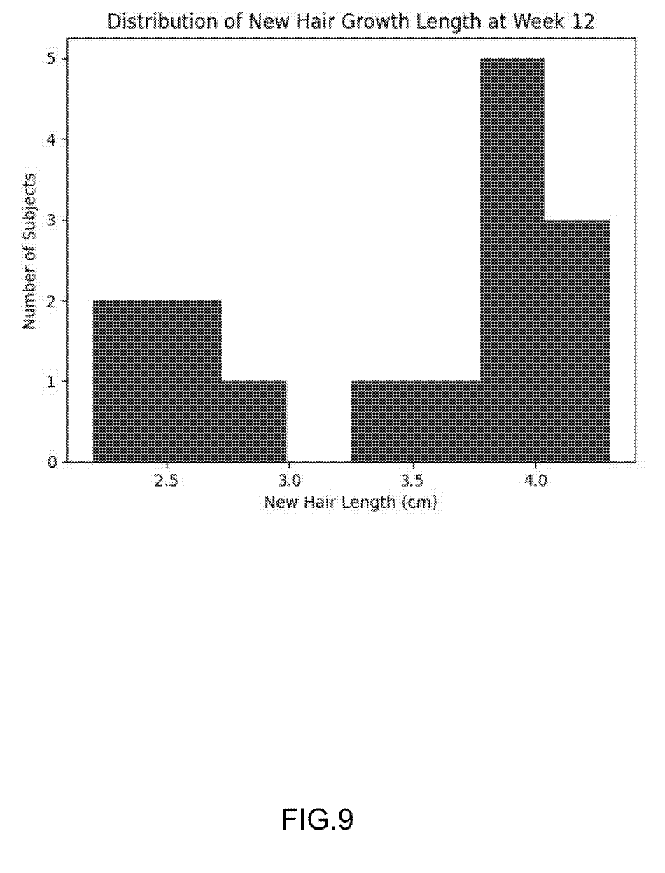

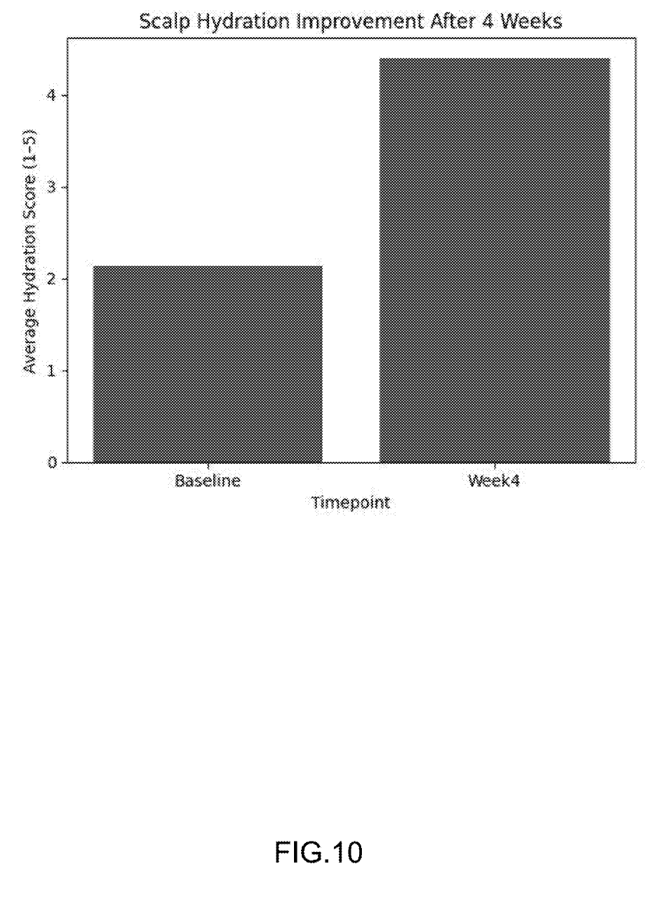

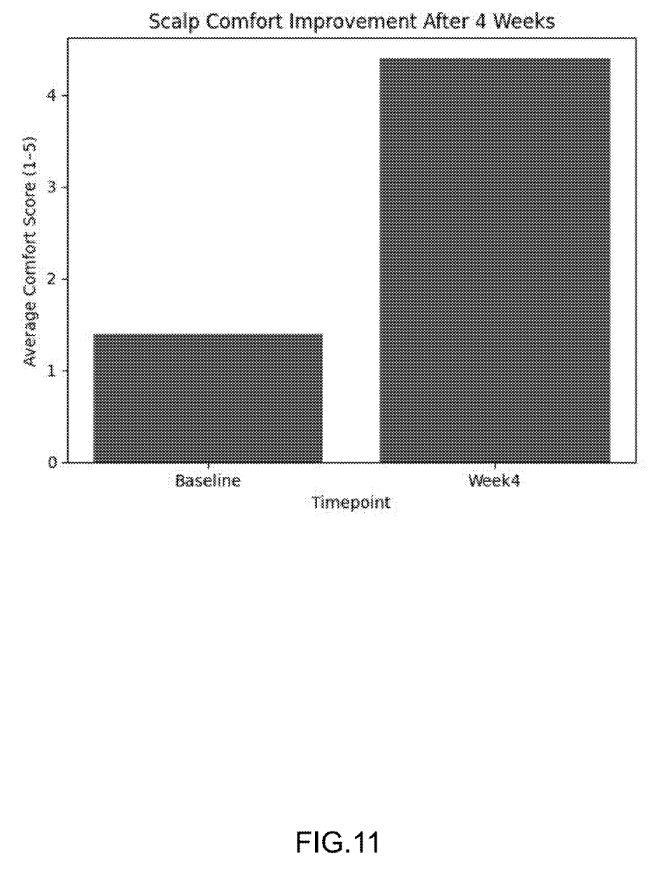

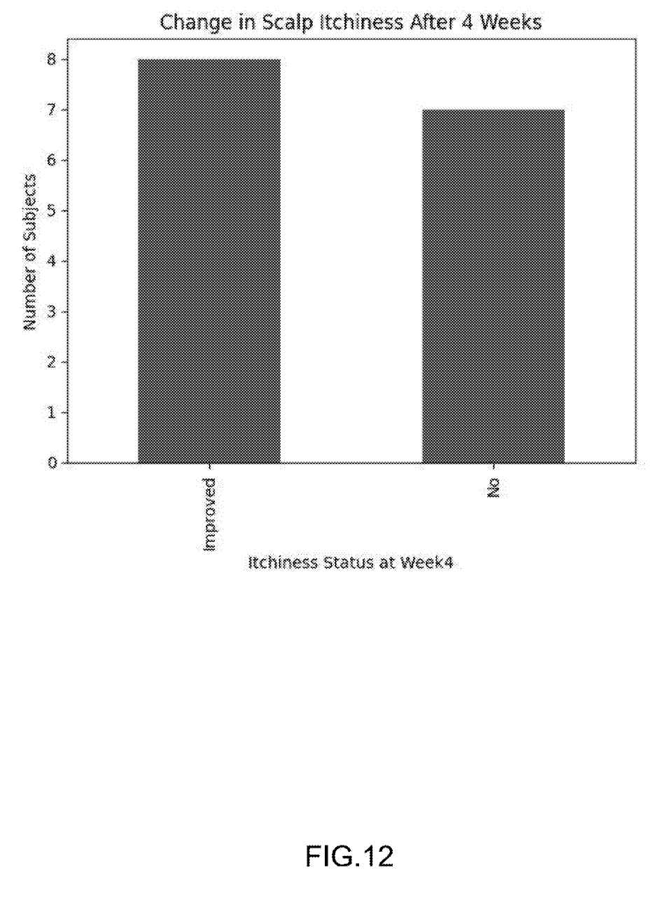

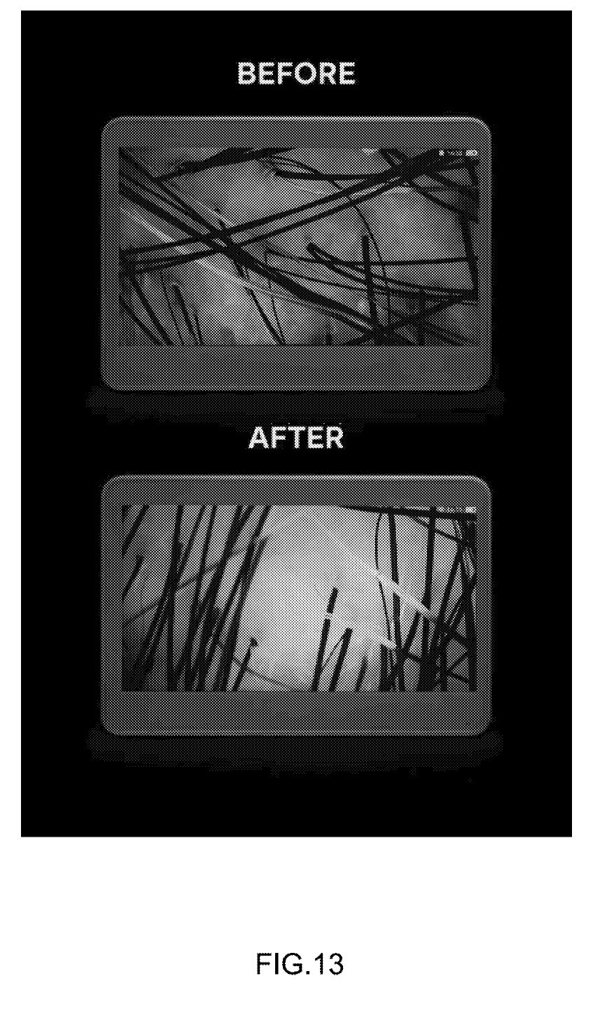

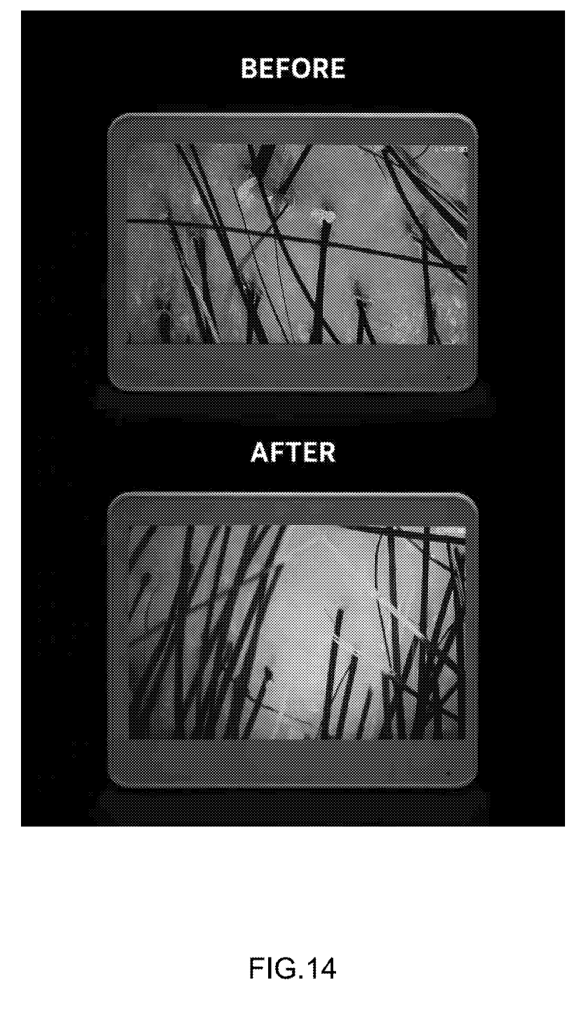

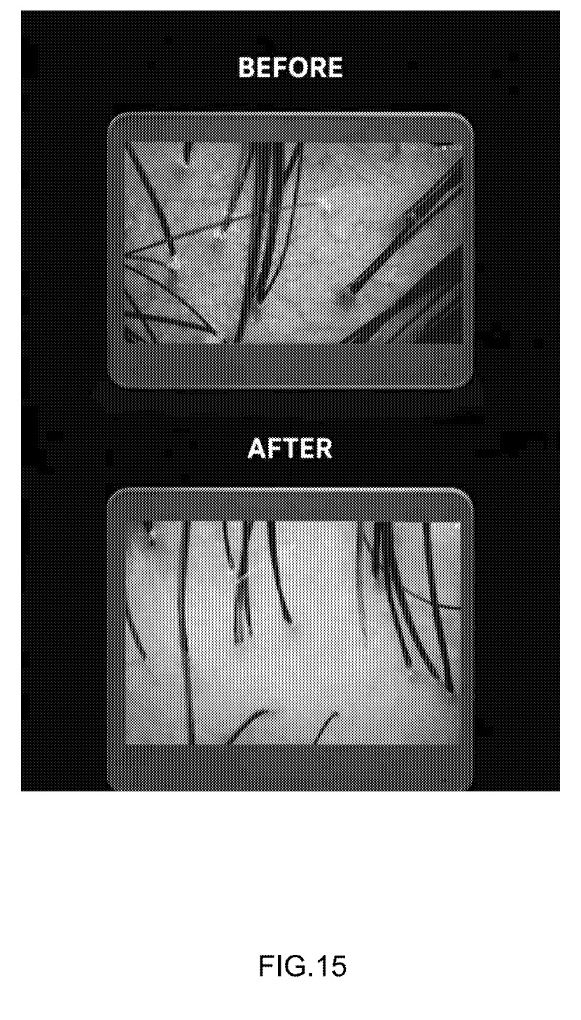

Please refer to FIGS. 1-15. The present disclosure aims to address the shortcomings of existing cleaning and care products, specifically including the following:
1. how to achieve the retention of effective ingredients in hair follicle units and adjacent areas within a short contact time of rinse type products, and form sustained functional support signals;
2. how to construct a collaborative system between shampoo and conditioner, break functional barriers, and achieve closed-loop care of cleaning, sedimentation, stability, and efficiency enhancement;
3. how to balance the effectiveness and mildness of the product, while increasing the loading of active ingredients, ensuring low irritation, low risk, and suitability for long-term daily use and special populations, such as sensitive muscles and after hair transplantation;
4. how to achieve visual improvement of scalp appearance state, such as erythema, hair follicle opening cleanliness, and oil/porphyrin deposition, through nonpharmacological means, and supporting hair follicle health and hair quality improvement.

To achieve the above objectives, the present disclosure provides a collaborative care method for scalp hair follicles and strands, including using collaboratively rinsing shampoo and conditioner, which achieve the retention and sustained effect of effective ingredients through a structural formula design and deposition mechanism. The specific technical scheme is as follows:

the shampoo and conditioner form a synergistic method of continuous use, the shampoo is responsible for cleaning scalp hair follicles, improving hair follicle permeability, and achieving initial deposition of effective ingredients; as a deposition amplifier and stabilizer, hair conditioner fixes and co deposits effective ingredients through a film-forming structure, reducing loss rate, stabilizing scalp microenvironment, and extending functional support time window; the two work together to achieve a closed-loop care of cleaning, deposition, stability and enhancement.

Rinse type shampoo formula system [weight percentage]
1. water-based matrix: purified water 40-65%;
2. mild surfactant agent: a total amount is 10-35%, including an anionic surfactant (disodium laureth sulfosuccinate 10-25%, sulfosuccinate 0-5%), a nonionic surfactant (decyl glucoside 5-15%, coco-glucoside 1-6%);
3. thickening and structural modifiers: sodium chloride 2-6%, hydroxyethyl cellulose 0.1-1%, PEG-150 pentaerythritol stearate 0.5-5%;
4. follicle support complex: a total amount of 0.5-10%, including olfactory receptor agonist (Sandalore 0.1-1%), metabolic stimulant (caffeine 0.05-1%), peptide and plant complex (biotinoyl tripeptide-1, apigenin, and oleanolic acid (supplied as Procapil or equivalent) 0.5-5%, saw palm extract 0.1-2%, white willow bark extract 0.0001-0.05%, rosemary extract 0.001-0.1%), vitamins and co factors (biotin 0.1-1%, pyridoxine 0.05-0.5%), and scalp-soothing or barrier-support agents (allantoin or derivatives thereof 0.001-2%);
5. conditioning and sedimentation promotion system: cationic polymer (polyquaternium-80 0.5-2%, polyquaternium-51 0.1-0.2%), moisturizer (glycerin 1-6%, PCA sodium 0.05-1%), and one or more deposition-promoting or conditioning amide compounds (dihydrogenated tallow phthalamic acid amide 0.1-8%), and skin-conditioning lipid carrier (caprylic/capric triglyceride 0.01-10%);
6. microbial regulator: leuconostoc/radish root fermentation filtrate 0.1-1%;
7. preservation system: a total amount of 0.05-2%, including 0.5-1% of phenoxyethanol, 0.01-0.5% of ethylhexylglycerin, 0.1-1% of sodium benzoate, and 0.01-0.5% of 1,2-hexanediol;
8. sensory regulating ingredient: essential oils (bergamot, etc.,), 0.1-1%;
9. pH regulator: adjusting a pH of the system to 5.0-5.8.

Hair conditioner formula system [weight percentage]
1. water-based matrix: water 40-85%;
2. film formation and stabilization system: amino functional silicone oil (aminoethylaminopropyl polydimethylsiloxane) 0.5-20%, fatty alcohol system (cetearyl alcohol, and docosyl alcohol) 2-20%, non-ionic surfactants (coco-glucoside) 0.1-5%, and emulsifying or stabilizing agents (potassium cetyl phosphate or derivatives thereof) 0.1-3%;
3. cation conditioning system: a total amount of 0.05-7%, including quaternary ammonium salts (Quaternium-80) and polyquaternium;
4. hydration and moisturizing system: hydration agent (glycerin, butanediol, etc.) 0.5-15%, natural oil and lipid moisturizer (including coconut oil, shea butter, argan oil, jojoba oil, caprylic/capric triglyceride, and/or squalane) 0.01-15%;
5. hair follicle support enhancing ingredients: olfactory receptor agonist (Sandalore 0.1-1%), biotinoyl tripeptide-1, apigenin, and oleanolic acid (supplied as Procapil or equivalent) 0.001-5%, peptides, plant lipid complexes 0.0001-10%, hydrolyzed collagen 0.01-10%, and one or more vitamin or antioxidant components 0.01-2%;
6. scalp soothing ingredient: plant extract (saw palmetto, celery seeds, willow bark extract, etc.) 0.0001-5%;
7. preservation and sensory system: preservation system 0.05-2%, aromatic compounds and sandalwood derivatives 0.001-2%;
8. pH regulator: adjusting a pH of the system to 4.0-6.0.

This method achieves effective ingredient retention after rinsing through the synergistic effect of the following three mechanisms:
1. cation deposition and carrying mechanism: cationic conditioning polymers and quaternary ammonium salts absorb onto negatively charged hair follicle orifice plug and hair roots, carrying active ingredients for co deposition;
2. lipid and surfactant composite deposition mechanism: mild surfactants form transient composite phases with lipid systems during washing and dilution, enriching hydrophobic/semi hydrophobic active ingredients;
3. geometric retention effect of hair follicle opening: funnel-shaped hair follicle opening structure combines with the microstructure of sebum and follicle orifice plug, reducing the erosion of sedimentary components by water flow.

Usage method:
1. applying an effective amount of shampoo composition to the moist scalp and massaging for 3-5 minutes to ensure that the hair follicles being fully in contact;
2. rinsing with 38-40 ℃ warm water until there is no residue;
3. taking the corresponding hair conditioner and applying it evenly to the scalp to the ends of the hair; massaging the hair root area for about 10 minutes;
4. gently rinsing with warm water until there is no sticky sensation at the ends of the hair, while retaining the protective layer of the scalp and hair roots;
5. using daily or every other day, with a continuous usage period of no less than 4 weeks, and a long-term care period of up to 6 months or more.

The above displays and describes the basic principles, main features, and advantages of the present disclosure. It is obvious to those skilled in the art that the present disclosure is not limited to the details of the exemplary embodiments described above, and can be implemented in other specific forms without departing from the spirit or basic features of the present disclosure. Therefore, from any point of view, the embodiments should be regarded as exemplary and non-limiting, and the scope of the present disclosure is limited by the appended claims rather than the above description. Therefore, it is intended to encompass all variations falling within the meaning and scope of the equivalent elements of the claims within the scope of the present disclosure.

Example 1: Low risk daily care method, suitable for sensitive scalp/long-term daily use

1. Shampoo formula [weight percentage]

Purified water 57.8%, disodium laureth sulfosuccinate 15%, decyl glucoside 8%, coco-glucoside 3%, sodium chloride 3%, PEG-150 pentaerythritol stearate 1.5%, glycerol 4%, PCA sodium 0.3%, polyquaternium-80 1%, polyquaternium-51 0.05%, hydroxyethyl cellulose 0.4%, Procapil 2%, saw palm extract 0.8%, caffeine 0.3%, biotin 0.2%, pyridoxine 0.1%, hydrolyzed collagen 0.5%, allantoin 0.2%, Sandalore 0.2%, bergamot essential oil 0.3%, leuconostoc /radish root fermentation filtrate 0.3%, white willow bark extract 0.02%, rosemary extract 0.03%, phenoxyethanol 0.6%, ethylhexylglycerin 0.1%, sodium benzoate 0.2%, and 1,2-hexanediol 0.1%.

2. Hair conditioner formula [weight percentage]

Water 68.5%, aminoethylaminopropyl polydimethylsiloxane 6.15%, cetearyl alcohol 3%, cetostearyl alcohol 2%, docosyl alcohol 1%, polyquaternium-80 2.2%, polyquaternium-51 0.3%, glycerol 5%, coco-glucoside 2.5%, butanediol 3%, jojoba oil 1.5%, shea butter 1%, peptide complex 0.5%, saw palmetto extract 0.2%, celery seed extract 0.1%, biotin 0.05%, tocopherol 0.05%, hydrolyzed collagen 1%, phenoxyethanol 0.3%, ethylhexylglycerin 0.05%, Sandalore 0.1%, caprylic/capric triglyceride 1%, and procapil-class compound 0.5%.

3. Preparation process

shampoo: stage A [aqueous phase]: heating purified water to 60 ℃, adding hydroxyethyl cellulose to disperse and hydrating for 20 minutes to gel shape;

stage B [surfactant]: cooling down to 45 ℃, low shearing [300rpm], adding anionic and nonionic surfactants, stirring for 15 minutes, gradually adding sodium chloride to adjust viscosity;

stage C [hydration and cationic polymer]: adding glycerol, PCA sodium, and quaternary ammonium salt, stirring for 10 minutes;

stage D [bioactive ingredient]: cooling down to 38 ℃, adding Procapil, plant extract, vitamins, and protein, and stirring for 15 minutes;

stage E [preservation and microbial regulation]: adding preservative and fermentation filtrate, stirring for 5 minutes;

stage F [sensory component]: cooling down to 30 ℃, adding Sandalore and essential oil, stirring for 10 minutes;

stage G: adjusting the pH to 5.2, homogenizing at 1500rpm for 5 minutes, degasifying for 10 minutes, cooling to 25 ℃, and then filling.

Hair conditioner: step 1 [aqueous phase]: heating water to 75 ℃, adding glycerol, butanediol, and water-soluble polymer, stirring for 20 minutes until completely hydrated;

step 2 [oil phase]: adding fatty alcohol, jojoba oil, shea butter oil, and amino silicone oil to an independent container, heating to 75 ℃, and melting uniformly;

step 3 [emulsification]: slowly injecting the oil phase into the aqueous phase under high-speed shearing at 3000rpm, and emulsifying for 15 minutes;

step 4 [cooling and adding active ingredient]: cooling down to 42 ℃, adding peptide complexes, plant extract, vitamin, and hydrolyzed collagen, and stirring for 10 minutes;

step 5 [conditioning optimization]: adding the cationic conditioning agent and stirring for 15 minutes;

step 6 [finished product adjustment]: adding preservative agent and fragrances, adjusting a pH to 4.8, homogenizing at 2000rpm for 5 minutes, removing bubbles, and then filling.

4. Effect verification

selecting 30 sensitive scalp subjects with mild erythema and hair shedding, and using the method of this example daily for 8 consecutive weeks:

hair shedding: after 2 uses, the average hair shedding decreased from 105 roots/time to 52 roots/time [↓ 50.5%], and after 4 weeks, it decreased to 32 roots/time [↓ 69.5%];

scalp condition: after 4 weeks, the disappearance rate of scalp erythema is 83%, the clarity of hair follicle opening is improved by 35%, and the incidence of dandruff is reduced from 60% to 10%;

safety: no subjects experienced irritation or discomfort, and the scalp barrier function [transcutaneous water diversion loss value] decreased by 28%.

Example 2: Post transplant caring care method, suitable for scalp of 1-3 months after surgery

1. Shampoo formula [weight percentage]

Purified water 60.9%, disodium laureth sulfosuccinate 10%, decyl glucoside 10%, coco-glucoside 4%, sodium chloride 2.5%, PEG-150 pentaerythritol stearate 1%, glycerol 5%, PCA sodium 0.5%, polyquaternium-80 0.8%, polyquaternium-51 0.1%, hydroxyethyl cellulose 0.3%, Procapil 1.5%, saw palm extract 0.5%, caffeine 0.2%, biotin 0.1%, pyridoxine 0.08%, hydrolyzed collagen 0.8%, allantoin 0.3%, Sandalore 0.1%, lavender essential oil 0.2%, leuconostoc /radish root fermentation filtrate 0.5%, white willow bark extract 0.01%, rosemary extract 0.01%, phenoxyethanol 0.4%, ethylhexylglycerin 0.05%, sodium benzoate 0.1%, and 1,2-hexanediol 0.05%.

2. Hair conditioner formula [weight percentage]

Water 72.5%, aminoethylaminopropyl polydimethylsiloxane 3.3%, cetearyl alcohol 2%, cetostearyl alcohol 1.5%, docosyl alcohol 0.5%, coco-glucoside 2%, polyquaternium-80 0.8%, polyquaternium-51 0.2%, glycerol 5.5%, butanediol 3%, squalane 1.5%, ceramide 0.8%, peptide complex 0.8%, procapil-class compound 2.2%, willow bark extract 0.3%, rosemary extract 0.7%, biotin 0.03%, tocopherol 0.07%, hydrolyzed collagen 0.5%, phenoxyethanol 0.2%, sandalore 0.3%, ethylhexylglycerin 0.1%, caprylic/capric triglyceride 0.5%, and bergamot fruit oil 0.7%.

3. Preparation process

shampoo: an aqueous phase temperature in stage A is controlled at 55 ℃, and a hydration time of hydroxyethyl cellulose is extended to 25 minutes;

after adding surfactant in stage B, a stirring speed should be reduced to 250rpm to avoid a formation of bubbles;

a temperature of adding bioactive ingredient in stage D should be controlled below 35 ℃ to ensure thermal stability and stability;

remaining steps are the same as in Example 1.

Hair conditioner: step 1: heating the aqueous phase to 70 ℃ and dispersing it with butanediol before adding ceramide;

step 3: reducing an emulsification shear speed to 2500rpm to avoid excessive stimulation of the components;

step 4: cooling to below 40 ℃ and adding the active ingredient;

remaining steps are the same as in Example 1.

4. Effect verification

Selecting 20 subjects who have undergone hair transplant surgery for one week and using the method of this example every other day for 12 consecutive weeks:

scalp recovery: average time for complete disappearance of postoperative erythema is 14 days, with significant relief of itching and no cases of postoperative infection;

hair follicle survival rate: after 12 weeks, the survival rate of hair follicles in the hair transplant area has increased significantly compared to a conventional care group;

scalp comfort: after 4 weeks of surgery, the scalp is well moisturized without dryness or tightness, and the pain caused by mechanical pulling disappears during combing.

Example 3: Enhanced hair follicle care method, suitable for individuals with sparse hair or insufficient hair follicle vitality

1. Shampoo formula [weight percentage]

Purified water 46.3%, disodium laureth sulfosuccinate 17.75%, decyl glucoside 8%, coco-glucoside 4%, sodium chloride 3.5%, PEG-150 pentaerythritol stearate 1.5%, glycerol 2.5%, PCA sodium 0.8%, polyquaternium-80 1.0%, polyquaternium-51 0.15%, hydroxyethyl cellulose 0.6%, Procapil 4%, saw palm extract 1.5%, caffeine 0.8%, biotin 0.5%, pyridoxine 0.2%, hydrolyzed collagen 1.2%, allantoin 0.4%, Sandalore 0.8%, bergamot essential oil 0.6%, leuconostoc /radish root fermentation filtrate 0.8%, caprylic/capric triglyceride 1%, white willow bark extract 0.04%, rosemary extract 0.06%, phenoxyethanol 0.8%, ethylhexylglycerin 0.3%, sodium benzoate 0.6%, and 1,2-hexanediol 0.3%.

2. Hair conditioner formula [weight percentage]

Water 58.9%, aminoethylaminopropyl polydimethylsiloxane 10%, cetearyl alcohol 4%, cetostearyl alcohol 3%, docosyl alcohol 2%, polyquaternium-80 2.5%, polyquaternium-51 0.5%, glycerol 3%, butanediol 2%, argan oil 2%, shea butter oil 1.5%, peptide complex 3%, saw palm extract 0.8%, celery seed extract 0.4%, biotin 0.1%, tocopherol 0.1%, hydrolyzed collagen 2%, phenoxyethanol 0.4%, ethylhexylglycerin 0.1%, caprylic/capric triglyceride 1%, procapil-class compound 2%, and Sandalore 0.7%.

3. Preparation process

shampoo: a stirring time of the bioactive ingredient in stage D is extended to 20 minutes to ensure uniform dispersion of high concentration ingredient;

after adding the sensory component in stage F, stirring for 15 minutes to ensure uniform distribution of the olfactory receptor agonist;

remaining steps are the same as in Example 1.

Hair conditioner: step 2: adding argan oil to the oil phase and stirring for 10 minutes until completely blended; step 4: after adding the peptide complex, keeping it at 40 ℃ and stirring for 15 minutes to enhance its activity stability; remaining steps are the same as in Example 1.

4. Effect verification

Selecting 25 subjects with sparse hair, with a part line width ≥ 3mm, and using the method of this example daily for 16 consecutive weeks:

hair density: after 12 weeks, the average part line width decreased to 1.8mm, and the appearance of hair density improved;

new hair growth: after 16 weeks, the average length of new growth reaches 3.8cm, with 76% of new growth having a diameter ≥ 0.08mm [coarse hard type];

reversibility verification: after 4 weeks of discontinuation, the hair shedding phenomenon significantly increased, and the part line width expanded to 2.3 mm; after 4 weeks of reuse, the amount of hair shedding significantly decreased, and the part line width reduced to 1.9 mm, with product dependent effects.

Example 4: Brand extended care method, shampoo, conditioner, scalp essence

1. Composition

Shampoo: same formula as Example 3;

Hair conditioner: same formula as Example 3;

Scalp essence formula [weight percentage]: water 88.3%, glycerin 5%, butanediol 3%, Procapil 2%, Sandalore 0.5%, caffeine 0.3%, biotin 0.1%, sodium hyaluronate 0.2%, panthenol 0.3%, phenoxyethanol 0.2%, ethyl hexyl glycerol 0.05%, and sandalwood essential oil 0.05%.

2. Usage method

On the basis of the use method in Example 3, the following steps were added: after washing the hair conditioner, pressing with a towel to dry the scalp, taking 3-5 ml of scalp essence and evenly smearing it on the hair root area, massaging for 1 minute until it is absorbed, without rinsing.

3. Effect verification

Selecting 15 subjects and using this extension method for 12 consecutive weeks:

synergistic effect: hair density improvement rate is 25% higher than using the washing and care combination alone, and the new hair growth rate is increased by 20%;

scalp condition: cleanliness of hair follicle openings has increased by 45%, oil secretion has decreased by 50%, and the maintenance rate of scalp microecological balance has reached 90%.

Example 5: Hair follicle channel opening type hair care method, suitable for individuals with seborrheic alopecia/hair follicle blockage

1. freeze-drying pretreatment solution formula [weight percentage]

Mannitol [freeze-drying protectant] 5%, salicylic acid 0.5%, disodium EDTA 0.1%, trehalose [stabilizer] 2%, sodium hyaluronate [hydration and soothing] 0.2%, arginine [pH regulator] 0.3%, purified water 91.9% [pre-freeze-drying system, moisture content ≤ 3% after freeze-drying].

2. Preparation process

step 1 [dissolve and mix]: taking purified water, adding mannitol, trehalose, and disodium EDTA, stirring until completely dissolved at 40 ℃;

step 2 [pH adjustment]: adding arginine and adjusting a pH of the system to 4.5-5.0 to avoid irritation of the scalp by salicylic acid;

step 3 [active ingredient addition]: cooling down to 30 ℃, slowly adding salicylic acid and sodium hyaluronate, stirring for 15 minutes until homogeneous;

step 4 [filtration and sterilization]: filtering with a 0.22 μm microporous membrane to remove impurities and microorganisms;

step 5 [freeze drying molding]: dividing the filtrate into penicillin bottles (2g per bottle), placing them in a freeze dryer, pre freezing to -40 ℃ and keeping warm for 2 hours, then sublime drying (vacuum degree 10Pa, temperature gradient: -30 ℃ → 0 ℃, heating rate 5 ℃/h, keeping warm for 8 hours), finally analyzing and drying (temperature 25 ℃, keeping warm for 4 hours), sealing and obtaining loose freeze-dried powder.

3. Complete usage method [adding preprocessing steps on the basis of the original method]

Taking 1 bottle of freeze-dried pretreatment solution, adding 5ml of 38 ℃ warm water, shaking until completely dissolved;

applying evenly to dry scalp, massaging a concentrated area (the top of the head and vertex) of hair follicles with fingertips for 1 minute, focusing on the hair follicle opening;

no need to rinse, applying shampoo directly according to the original usage method, the subsequent steps of massage, rinsing, and conditioner use remain unchanged;

using once a day, with a continuous usage period of no less than 4 weeks; after hair follicle blockage has improved, it can be changed to use every other day.

4. Effect verification

Selecting 20 subjects with seborrheic alopecia, with a positive rate of ≥ 70% for follicular opening follicle orifice plugs and a hair shedding rate of ≥ 80 hairs/time; using pre-treatment solution in combination with the washing and care method described in Example 3 for 12 consecutive weeks;

component absorption rate: through high-performance liquid chromatography detection, the deposition amount in hair follicles increased by 52% compared to the group without pre-treatment solution, and the caffeine deposition amount increased by 48%;

improvement of hair follicle blockage: after 8 weeks, the positive rate of hair follicle opening follicle orifice plug decreased from 75% to 23%, and the smoothness of hair follicle opening increased by 65% under the microscope;

improvement of hair shedding: after 4 weeks, the average amount of hair shedding decreased from 92 roots/time to 38 roots/time [↓ 58.7%], and after 12 weeks, it decreased to 25 roots/time [↓ 72.8%], significantly better than using the hair care method alone [12 weeks hair shedding amount ↓ 55.3%];

safety: no subjects experienced scalp pain or redness, and there was no significant increase in transdermal water loss, the mildness meets the standards for sensitive scalp use.

Although the embodiments of the present disclosure have been shown and described, it will be understood by those skilled in the art that various changes, modifications, substitutions, and variations can be made to these embodiments without departing from the principles and spirit of the present disclosure. The scope of the present disclosure is limited by the appended claims and their equivalents.

What is claimed is:

1. A collaborative care method for scalp hair follicles and strands, comprises using collaboratively rinse shampoo and conditioner;
the shampoo comprises a mild surfactant, a hair follicle support complex, a cationic conditioning polymer, and a microbiome regulator;
the conditioner comprises amino functional silicone oil, a fatty alcohol system, a cationic conditioning system, and a hair follicle support enhancing ingredient; and the method achieves a retention of effective ingredients in hair follicle units and adjacent areas after washing through a synergistic mechanism of cation deposition and carrier, lipid - surfactant composite deposition, and geometric retention at hair follicle;
wherein the retention is achieved specifically by (i) cationic polymer co-deposition on negatively charged keratin, (ii) transient lipid-surfactant composite phases during rinse, and (iii) geometric entrapment in the funnel-shaped follicle opening.

2. The collaborative care method for scalp hair follicles and strands according to claim 1, wherein the shampoo comprises the following component by mass of water-based matrix: 40-65%, mild surfactant agent: 10-35%, which comprising an anionic surfactant and a nonionic surfactant; thickening and structural modifiers that comprises sodium chloride 2-6%, hydroxyethyl cellulose 0.1-1%, PEG-150 pentaerythritol stearate 0.5-5%; follicle support complex: 0.5-10%, comprising olfactory receptor agonist, metabolic stimulant, peptide and plant complex, vitamins and co factors, and scalp-soothing or barrier-support agents, optionally including allantoin or derivatives thereof; conditioning and sedimentation promotion system comprising cationic polymer and moisturizer; deposition-promoting or conditioning amide compounds; skin-conditioning lipid carrier, microbial regulator; preservation system: 0.05-2%, comprising phenoxyethanol, ethylhexylglycerin, sodium benzoate, and 1,2-hexanediol; sensory regulating ingredient: 0.1-1%; and a pH regulator.

3. The collaborative care method for scalp hair follicles and strands according to claim 1, wherein the conditioner comprises the following component by mass of water-based matrix: 40-85%; film formation and stabilization system: comprising 0.5-20% of amino functional silicone oil, 2-20% of fatty alcohol system; cation conditioning system: 0.01-7%, comprising quaternary ammonium salts and polyquaternium; hydration and moisturizing system: comprising 0.5-15% of hydration agent and 0.1-15% of natural oil and lipid moisturizer; hair follicle support enhancing ingredients comprising olfactory receptor agonist, biotinoyl tripeptide-1, apigenin, and oleanolic acid, peptide complexes, plant lipid complexes, caprylic/capric triglyceride, and hydrolyzed collagen; scalp soothing ingredient: comprising plant extract; preservation and sensory system; emulsifying or stabilizing agents, non-ionic surfactants, vitamins or antioxidant components, and a pH regulator.

4. The collaborative care method for scalp hair follicles and strands according to claim 1, wherein a total amount of the mild surfactant in the shampoo is 10-35wt%, and the mild surfactant comprises 3-25wt% of anionic surfactant and 1-20wt% of nonionic surfactant;
a total amount of the hair follicle support complex is 0.5-10wt%, and the hair follicle support complex comprises 0.01-2wt% of olfactory receptor agonist, peptide component, plant extract, and vitamin derivative.

5. The collaborative care method for scalp hair follicles and strands according to claim 1, wherein the amino functional silicone oil has a content of 0.5-20 wt% of the hair conditioner, a total amount of the fatty alcohol system is 2-20wt%, a total amount of the cationic conditioning system is 0.05-7wt%, and a pH is 4.0-6.0.

6. The collaborative care method for scalp hair follicles and strands according to claim 1, wherein raw materials of the shampoo and conditioner have been screened with an Environmental Working Group database, (EWG) score of 0-2 and basically do not contain high-risk or endocrine disrupting ingredients.

7. The collaborative care method for scalp hair follicles and strands according to claim 1, wherein the shampoo is applied to the scalp and massaged for 3-5 minutes before rinsing, the hair conditioner is applied and massaged for about 10 minutes before gently rinsing, and the hair root is smeared with scalp essence and absorbed.

8. The collaborative care method for scalp hair follicles and strands according to claim 1, wherein the method improves appearance of the scalp, reduces hair shedding, and enhances hair density through a synergistic effect of cleaning, sedimentation, stabilization, and enhancement.

9. The collaborative care method for scalp hair follicles and strands according to claim 7, wherein the essence liquid comprises water, glycerin, butanediol, biotinoyl tripeptide-1, apigenin, and oleanolic acid, supplied as Procapil or equivalent, Sandalore, caffeine, biotin, sodium hyaluronate, panthenol, phenoxyethanol, and ethyl hexyl glycerol.

10. The collaborative care method for scalp hair follicles and strands according to claim 1, further comprising a freeze-dried pretreatment solution that is used synergy; the pretreatment solution is a freeze-dried powder containing low concentration salicylic acid 0.5wt%, disodium EDTA 0.1wt%, and freeze-drying protectant;
the pretreatment solution is dissolved in 38 ℃ warm water before use, applied to the scalp and massaged for 1 minute before shampooing;
the pretreatment solution is configured to gently dissolve hair follicle orifice plugs, open hair follicle channels, and greatly improve an absorption rate of effective ingredients in a subsequent shampoo and it is suitable for people with hair follicle blockage type hair shedding.
